(19) United States
(12) Patent Application Publication
(10) Pub. No.: US 2026/0257060 A1
(43) Pub. Date: Sep. 3, 2026

(54) AESTHETIC TREATMENT DEVICE

(71) Applicant: HIRONIC CO., LTD., Yongin-si, Gyeonggi-do (KR)

(72) Inventor: Jin Woo LEE, Yongin-si, Gyeonggi-do (KR)

(73) Assignee: HIRONIC CO., LTD., Yongin-si, Gyeonggi-do (KR)

(21) Appl. No.: 19/165,105

(22) PCT Filed: Mar. 14, 2024
(86) PCT No.: PCT/KR2024/003256

§ 371 Date: Sep. 12, 2025

(30) Foreign Application Priority Data
Mar. 15, 2023 (KR) 10-2023-0033909

(51) Int. Cl. A61N 1/32; A61N 1/36; A61N 1/40; A61N 7/00; A61N 7/02

(52) U.S. Cl. CPC A61N 1/328; A61N 1/36034; A61N 1/40; A61N 7/02; A61N 2007/0034

(57) ABSTRACT

An aesthetic treatment device includes a handpiece that is held by a practitioner for treatment, a cartridge that is detachably attached to the handpiece, a radio-frequency generator that is positioned at one end of the cartridge to selectively apply a first current and a second current to a treatment area of the subject's skin, a focused ultrasound generator that is positioned at one end of the cartridge adjacent to the radio-frequency generator to transmit focused ultrasound to the treatment area, and a controller that controls the operation of the radio-frequency generator and the focused ultrasound generator.

### TECHNICAL FIELD

The present invention relates to an aesthetic treatment device.

### BACKGROUND ART

Recently, interest in skin care and obesity treatment is increasing day by day, and various aesthetic medical devices for skin care and obesity treatment are being developed. For example, various aesthetic medical devices for face lifting, skin tightening, or lipolysis procedures are being developed.

Non-invasive procedures for skin care and obesity include methods using radiofrequency (RF), vibration massage, cryo-lipolysis, and focused ultrasound.

As described above, various non-invasive procedures can be selectively performed depending on the patient's condition and needs, and research and development of aesthetic medical devices for each treatment are ongoing.

Prior art documents include Korean Patent No. 10-1574951 (published on Dec. 7, 2015).

### DISCLOSURE

### Technical Problem

It is an object of the present invention to provide an aesthetic treatment device in which a controller controls a radio-frequency generator and a focused ultrasound generator arranged at one end of a cartridge to generate a first current, a second current, and focused ultrasound to a treatment area according to a first treatment period, a second treatment period and a third treatment period.

### Technical Solution

In accordance with an aspect of the present invention, the above and other objects can be accomplished by the provision of an aesthetic treatment device comprising: a handpiece that is held by a practitioner for treatment; a cartridge that is detachably attached to the handpiece; a radio-frequency generator that is positioned at one end of the cartridge to selectively apply a first current and a second current to a treatment area of the subject's skin; a focused ultrasound generator that is positioned at one end of the cartridge adjacent to the radio-frequency generator to transmit focused ultrasound to the treatment area; and a controller that controls the operation of the radio-frequency generator and the focused ultrasound generator, wherein the controller is configured to control the radio-frequency generator to generate the first current during a first treatment period, and to generate the second current during a second treatment period following the first treatment period, and wherein the controller is configured to control the focused ultrasound generator to irradiate focused ultrasound to the treatment area between the first and second treatment periods, or during a third treatment period following the second treatment period, and the radio-frequency generator is configured to generate the first current and the second current having different magnitudes.

The controller is configured to control the operation of the radio-frequency generator and the focused ultrasound generator so that the first treatment period, the second treatment period, and the third treatment period are periodically repeated.

The controller is configured to control the radio-frequency generator and the focused ultrasound generator so that a penetration depth of the first current or the second current and an irradiation depth of the focused ultrasound are different from each other at the treatment area.

The controller is configured to control the focused ultrasound generator so that the focused ultrasound is irradiated to a depth of 1.5 mm to 4.5 mm from a surface of the skin.

The radio-frequency generator is configured to generate radio-frequency energy in the frequency range of 1 MHz to 10 MHz, and the focused ultrasound generator is configured to generate focused ultrasound energy in the frequency range of 1 MHz to 10 MHz

The aesthetic treatment device further comprises a position change detector for sensing at least one of movement, movement speed, movement acceleration, angular velocity, position, movement direction, inclination, and displacement of the handpiece or the cartridge.

### Advantageous Effects

An aesthetic treatment device according to the present invention has an advantageous effect that a controller controls a radio-frequency generator and a focused ultrasound generator arranged at one end of a cartridge to generate a first current, a second current, and focused ultrasound to a treatment area according to a first treatment period, a second treatment period and a third treatment period.

### DESCRIPTION OF DRAWINGS

FIG. 1 shows an aesthetic treatment device according to an embodiment of the present invention.

FIG. 2 shows control steps of the aesthetic treatment device according to an embodiment of the present invention.

### BEST MODE

The present invention is capable of various modifications and embodiments, some of which are illustrated in the drawings and described in detail in the descriptions. These drawings and detailed descriptions are not intended to limit the present invention to specific embodiments. It should be understood that all modifications, equivalents, and alternatives falling within ideas and technical scope of the present invention are included in this specification. In describing the present invention, detailed descriptions of related known technologies will be omitted if they are deemed to obscure the gist of the present invention.

Terms such as “first,” “second,” etc. may be used to describe various elements, but these elements should not be limited by these terms. These terms are just used to distinguish one element from another.

The terminology used herein is just used to describe specific embodiments and is not intended to limit the present invention. The singular forms “a,” “an,” and “the” include plural forms unless the context clearly indicates otherwise. In this specification, terms such as “include” or “have” are intended to indicate the presence of a feature, number, step, operation, element, part, or combination thereof described in the specification, and should be understood as not excluding in advance the possibility of the presence or addition of one or more other features, numbers, steps, operations, elements, parts, or combinations thereof.

Furthermore, the term “combination” does not only refer to direct physical contact between elements, but can also be used as a concept encompassing cases where another element is interposed between said elements, and the said elements are indirectly in contact with each other through the another element.

Hereinafter, an embodiment of an aesthetic treatment device 100 according to the present invention will be described in detail with reference to the drawings. When describing with reference to the drawings, identical or corresponding components or elements are assigned the same drawing numbers and redundant descriptions thereof will be omitted.

An aesthetic treatment device according to an embodiment of the present invention may include a handpiece 100, a cartridge 120, a radio-frequency generator 130, a focused ultrasound generator 140, and a controller 150. The handpiece 110 can be held by a practitioner for treatment. The cartridge 120 can be detachably attached to the handpiece 110. The radio-frequency generator 130 can be positioned at one end of the cartridge 120 to selectively apply a first current and a second current to a treatment area of the subject's skin. The focused ultrasound generator 140 can be positioned at one end of the cartridge 120 adjacent to the radio-frequency generator 130 to transmit focused ultrasound to the treatment area. The controller 150 may control the operation of the radio-frequency generator 130 and the focused ultrasound generator 140. The controller 150 may control the radio-frequency generator 130 to generate the first current during a first treatment period, and to generate the second current during a second treatment period following the first treatment period. Furthermore, the controller may control the focused ultrasound generator 140 to irradiate focused ultrasound to the treatment area between the first and second treatment periods, or during a third treatment period following the second treatment period.

In the aesthetic treatment device 100 according to an embodiment of the present embodiment, the controller 150 can control the radio-frequency generator 130 and the focused ultrasound generator 140 arranged at one end of the cartridge 120 to generate the first current, the second current, and focused ultrasound to the treatment area according to the first treatment period, the second treatment period and the third treatment period.

Wherein the controller 150 may control the radio-frequency generator 130 and the focused ultrasound generator 140 to generate a first current, a second current, or focused ultrasound in the first, second, and third treatment periods, respectively, thereby enhancing the skin lifting or fat dissolving effect achieved in the treatment area.

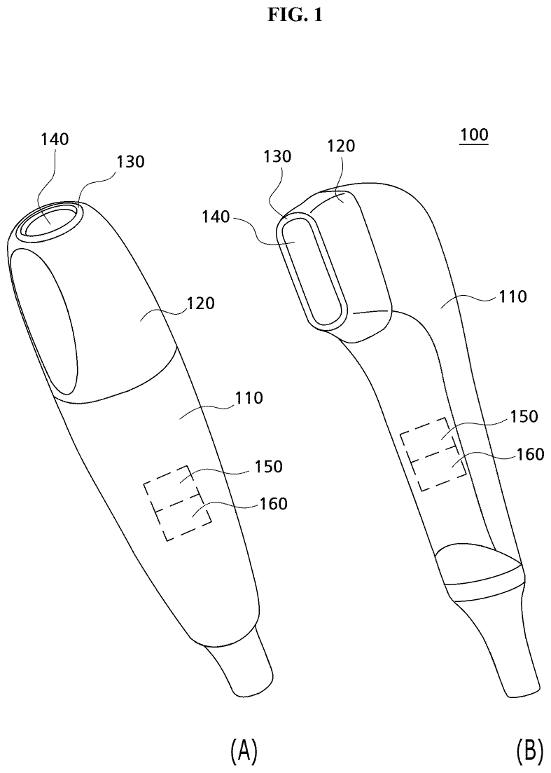

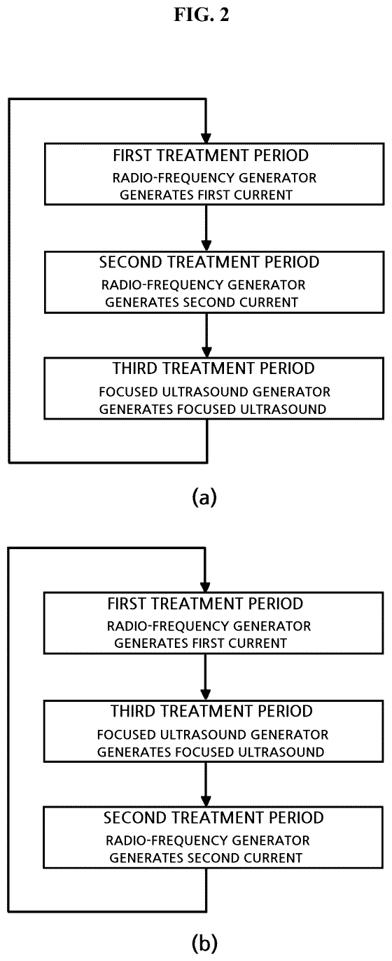

Hereinafter, with reference to FIGS. 1 and 2, each element of the aesthetic treatment device 100 according to an embodiment of the present invention and the operation control steps of the aesthetic treatment device 100 will be described.

As shown in FIG. 1, the handpiece 110 may be provided with a grip portion that allows the practitioner to grip it for treatment, and the cartridge 120 may be detachably attached to one side of the handpiece. More specifically, the handpiece 110 may be connected to a separately provided main device and configured to receive power and control signals from the main device.

As shown in FIG. 1(A), a cartridge 120 may be configured to be detachably attached to the distal end of the handpiece 110. Alternatively, as shown in FIG. 1(B), a cartridge 120 may be configured to be detachably attached to the side of the handpiece 110. Depending on the configuration of the handpiece 110, the structure of the cartridge 120 and the directions in which the radio-frequency generator 130 and the focused ultrasound generator 140 positioned within the cartridge 120 may be configured differently.

As shown in FIG. 1, the radio-frequency generator 130 is positioned at the end of the cartridge 120 to selectively apply a first current and a second current to the treatment area and to detect whether current is being applied. The focused ultrasound generator 140 is positioned adjacent to the radio-frequency generator 130 at one end of the cartridge 120 to irradiate focused ultrasound to the treatment area.

More specifically, the radio-frequency generator 130 may be positioned on the same surface as the focused ultrasound generator 140 and the cartridge 120, and may be positioned along the outer surface of the focused ultrasound generator 140.

Furthermore, the focused ultrasound generator 140 may be formed in a circular shape, as shown in FIG. 1(A), or may be configured as a linear structure comprised of linear and semicircular portions, as shown in FIG. 1(B). The radio-frequency generator 130 may be positioned along the periphery of the irradiation surface of the focused ultrasound generator 140.

The radio-frequency generator 130 may be formed as a single unit along the periphery of the focused ultrasound generator 140, or multiple units may be arranged along the periphery of the focused ultrasound generator 140.

As shown in FIG. 1, the controller 150 may control operations of the radio-frequency generator 130 and the focused ultrasound generator 140. More specifically, when the end of the cartridge 120 in which the radio-frequency generator 130 and the focused ultrasound generator 140 are disposed is positioned on the treatment area, the controller 150 may control operations of the radio-frequency generator 130 and the focused ultrasound generator 140 during the preset first treatment period, the second treatment period, and the third treatment period, respectively, to perform the skin care treatment.

As shown in FIG. 2, the controller 150 may control the radio-frequency generator 130 to generate a first current in the first treatment period, control the radio-frequency generator 130 to generate a second current in the second treatment period after the first treatment period, and control the focused ultrasound generator 140 to irradiate focused ultrasound to the treatment area between the first treatment period and the second treatment period, or after the second treatment period.

As shown in FIG. 2, the controller 150 may control the operation of the radio-frequency generator 130 and the focused ultrasound generator 140 so that the first to third treatment periods are periodically repeated.

In addition, the radio-frequency generator 130 may generate a first current and a second current having different magnitudes. The controller 150 may control the radio-frequency generator 130 to generate a first current of a preset intensity in the first treatment period, which is a preset time range, and may control the radio-frequency generator 130 to sequentially generate a preset second current with an intensity greater than the first current in the second treatment period, which is a preset time range.

The controller may allow the second current to apply greater electrical energy than the first current to transmit stronger and stronger electrical stimulation to the treatment area as it passes through the first treatment period and the second treatment period. As the temperature of the treatment area increases more and more, the treatment effect can be improved.

Additionally, as shown in FIG. 2, if the controller 150 operates the radio-frequency generator 130 in the first and second treatment periods, the temperature at the treatment area can be increased by radio-frequency energy, and if the focused ultrasound generator 140 operates in the third treatment period while the treatment area is heated, it can effectively reach the target temperature with a lower energy output.

Therefore, in the present embodiment of the present invention, the controller 150 may control the output of the radio-frequency generator 130 and the focused ultrasonic generator 140 based on the temperature of the treatment area.

The controller 150 may control the radio-frequency generator 130 and the focused ultrasound generator 140 such that the penetration depth of the first current or the second current and the transmission depth of the focused ultrasound are different from each other at the treatment area. The controller 150 may control the focused ultrasound generator 140 to irradiate the focused ultrasound to a depth within a range of 1.5 mm to 4.5 mm from the skin surface.

As described above, as the controller 150 controls the focused ultrasound generator 140, the aesthetic treatment device according to an embodiment of the present invention may perform a face lifting or skin tightening procedure by concentrating energy on the dermis layer, fascia layer, or SMAS layer located 1.5 mm to 4.5 mm from the skin surface to form a thermal lesion or thermal coagulation region.

The controller 150 may control the radio-frequency generator 130 and the focused ultrasound generator 140 so that the penetration depth of the first current or the second current and the transmission depth of the focused ultrasound are different from each other to generate heat overall in the upper and lower parts of a target point in the dermis of the treatment area.

Additionally, the radio-frequency generator 130 may generate radio-frequency energy in the frequency range of 1 MHz to 10 MHz, and the focused ultrasonic generator 140 may generate focused ultrasonic energy in the frequency range of 1 MHz to 10 MHz.

As shown in FIG. 1, the aesthetic treatment device according to an embodiment of the present invention may further include a position change detector 160 that detects at least one of movement, movement speed, movement acceleration, angular velocity, position, movement direction, slope, and displacement of the handpiece 110 or cartridge 120.

The controller 150 may control the operation of the radio-frequency generator 130 and the focusing ultrasonic generator 140 based on at least one of movement, movement speed, movement acceleration, angular velocity, position, movement direction, inclination, and displacement of the handpiece 110 or cartridge 120 sensed by the position change detector 160.

For example, if the controller determines that the handpiece 110 or cartridge 120 does not continue to move or that rapid movement occurs based on information provided by the position change detector 160, the controller 150 may stop the operation of the radio-frequency generator 130 and the focused ultrasound generator 140.

Although an embodiment of the present invention has been described above, a person skilled in the art may modify and change the present invention in various ways by adding, changing, deleting, or adding components to the scope of the present invention described in the claims, and this is also included within the scope of the present invention.

### INDUSTRIAL APPLICABILITY

An aesthetic treatment device according to the present invention may be used in industries related to skin care or obesity disease procedures by a non-invasive method using radio-frequency and focused ultrasound.

1. An aesthetic treatment device comprising:
a handpiece that is held by a practitioner for treatment;
a cartridge that is detachably attached to the handpiece;
a radio-frequency generator that is positioned at one end of the cartridge to selectively apply a first current and a second current to a treatment area of the subject's skin;
a focused ultrasound generator that is positioned at one end of the cartridge adjacent to the radio-frequency generator to transmit focused ultrasound to the treatment area; and
a controller that controls the operation of the radio-frequency generator and the focused ultrasound generator, wherein the controller is configured to control the radio-frequency generator to generate the first current during a first treatment period, and to generate the second current during a second treatment period following the first treatment period, and wherein the controller is configured to control the focused ultrasound generator to irradiate focused ultrasound to the treatment area between the first and second treatment periods, or during a third treatment period following the second treatment period.

2. The aesthetic treatment device according to claim 1, wherein the radio-frequency generator is configured to generate the first current and the second current having different magnitudes.

3. The aesthetic treatment device according to claim 1, wherein the controller is configured to control the operation of the radio-frequency generator and the focused ultrasound generator so that the first treatment period, the second treatment period, and the third treatment period are periodically repeated.

4. The aesthetic treatment device according to claim 1, wherein the controller is configured to control the radio-frequency generator and the focused ultrasound generator so that a penetration depth of the first current or the second current and an irradiation depth of the focused ultrasound are different from each other at the treatment area.

5. The aesthetic treatment device according to claim 4, wherein the controller is configured to control the focused ultrasound generator so that the focused ultrasound is irradiated to a depth of 1.5 mm to 4.5 mm from a surface of the skin.

6. The aesthetic treatment device according to claim 1, wherein the radio-frequency generator is configured to generate radio-frequency energy in the frequency range of 1 MHz to 10 MHz, and the focused ultrasound generator is configured to generate focused ultrasound energy in the frequency range of 1 MHz to 10 MHz

7. The aesthetic treatment device according to claim 1, further comprising a position change detector for sensing at least one of movement, movement speed, movement acceleration, angular velocity, position, movement direction, inclination, and displacement of the handpiece or the cartridge.
